(19) United States
(12) Patent Application Publication
(10) Pub. No.: US 2008/0140195 A1
(43) Pub. Date: Jun. 12, 2008

(54) MULTIFUNCTIONAL NANO-PROBE INTERFACE STRUCTURE FOR NEURAL PROSTHESES AND MANUFACTURING METHOD THEREOF

(75) Inventors: Huan-Chieh Su, Hsinchu (TW); Hsieh Chen, Hsinchu (TW); Hsin Chen, Hsinchu (TW); Yen-Chung Chang, Hsinchu (TW); Shih-Rung Yeh, Hsinchu (TW); Weileun Fang, Hsinchu (TW); Chien-Chung Fu, Hsinchu (TW); Shiang-Cheng Lu, Zhubei City (TW); Tri-Rung Yew, Hsinchu City (TW)

Correspondence Address: JIANQ CHYUN INTELLECTUAL PROPERTY OFFICE, 7 FLOOR-1, NO. 100, ROOSEVELT ROAD, SECTION 2, TAIPEI, 100, omitted

(73) Assignee: NATIONAL TSING HUA UNIVERSITY, Hsinchu (TW)

(21) Appl. No.: 11/775,878

(22) Filed: Jul. 11, 2007

(30) Foreign Application Priority Data
Nov. 8, 2006 (TW) 95141327

(51) Int. Cl. A61F 2/02

(52) U.S. Cl. 623/11.11; 977/742

(57) ABSTRACT

A novel multifunctional nano-probe interface is proposed for applications in neural stimulation and detecting. The nano-probe interface structure consists of a carbon nanotube coated with a thin isolation layer, a micro-electrode substrate array, and a controller IC for neural cell recording and stimulation. The micro-electrode substrate array contains wires connecting the carbon nanotube with the controller IC, as well as microfluidic channels for supplying neural tissues with essential nutrition and medicine. The carbon nanotube is disposed on the micro-electrode substrate array made by silicon, coated with a thin isolation layer around thereof, and employed as a nano-probe for neural recording and stimulation.

### CROSS-REFERENCE TO RELATED APPLICATION

This application claims the priority benefit of Taiwan application serial no. 95141327, filed Nov. 8, 2006. All disclosure of the Taiwan application is incorporated herein by reference.

### BACKGROUND OF THE INVENTION

1. Field of the Invention

The present invention relates to a multifunctional nano-probe interface structure and a manufacturing method thereof for applications in neural prostheses.

2. Description of Related Art

Since the activity of intracephalic neural cells is mainly achieved through electrical signals, based upon appreciating the operation of human brains, many mechanical instruments have been developed and applied to aid the functions of the nerve and muscle tissues disabled due to diseases or degraded due to old age.

In Nature, July, 2006 (seen L. R. Hochber, M. D. Serruya, G. M. Friehs, J. A. Mukand, M. Saleh, A. H. Caplan, A. Branner, D. Chen, R. D. Penn, J. P. Donoghue, Nature, 442:154-177 (2006)), a brain-machine interface (BMI) made by an Utah electrode array is reported. The research team of Hochber, et al. implanted the multi-electrode array (10×10 electrodes) into the brain of a quadriplegic patient having spinal cord injuries. After working together with a brain-machine interface containing suitable software and hardware, the patient is capable of utilizing the activity of the brain to directly execute some complex and accurate instrument controlled operations through a computer, without being trained for a long time. That is to say, in the future, the mechanical aids can help patients with brain injuries or degeneration to recover a part of the self-care abilities through the brain-machine interfaces.

The Utah electrode array (see Wim L. C. Rutten, Annu. Rev. Biomed. Eng. Vol. 4:407-452 (2002)) is formed by a pointed cone etched by doped silicon, and then, an isolation layer of Si₃N₄ is deposited on the silicon pointed cone, and finally, a metal, such as Pt and Ti, is deposited thereon after lithography process. This type of multi-electrode probed array records the activities of a plurality of neural cells and interprets the activities of the brain more sensitively, accurately, and rapidly, compared with electroencephalogram (EEG). Therefore, the implanted multiple electrode probe array will become the crucial technique for development of the brain-machine in the future.

However, various micro-electrode probe arrays currently developed still cannot reliably detect the activity of the neural cell for a long term, and cannot stimulate the neural tissues regionally and selectively, which are mainly due to the following disadvantages: (1) the size of the electrode manufactured by the micro-mechanical technique is still too large, which thus easily causes damages to the cell, or cannot stimulate and record a single cell, and the electrode and substrate have an insufficient elasticity, and thus likely being moved and falling off due to the movement of the human body and causing damages; (2) currently, most of the electrodes are metallic electrodes, which have a high impedance at a low frequency region, and except the action potential, the electrodes cannot sensitively detect other potential changes of the tissue; (3) the metal and the tissue are electrochemically reacted, so that the neural prostheses cannot identify whether the detected signal comes from the tissue or from the electrochemical reaction of the electrode itself, (4) the long-term implantation causes a partial inflammation or an immune-relevant reaction, so that the magnitude of output current should be continuously enhanced; otherwise, the detection sensitivity might be reduced.

Since the size of the carbon nanotube is small (can be less than 10 nm), the damages on the nerve can be minimized; on the other aspect, the carbon nanotube and the neural tissue are not electrochemically reacted due to the way of current conducting, so that the impedance of the electrode will be negligibly changed and no degradation on the measurement of neural activities will occur. In this way, the disadvantages of the metal electrode are avoided.

In U.S. Patent publication No. 20040182707A1, a method for manufacturing a nano-probe or a nano-electrode is disclosed, which is punctured into a cell to measure the potential property in the cell, and applied in biosensor to monitor the potential response of the cell, combined with the arrayed electrode and microfluidic channel design. The nano-probe is made of silicon, metal, and carbon nano-fiber (CNF), and the diameter of the probe is 50 nm to 1 μm.

### SUMMARY OF THE INVENTION

In the present invention, a carbon nanotube is taken as the nano-probe to puncture the cell, and through successfully combining with the micro-electrode array, it has a plurality of functions, such as being easily combined with microfluidic channel controlling circuit. Moreover, the diameter of the probe is getting to be less than 1 nm to 50 nm, and thus, besides causing less damage to the neural cell, the probe can also accurately and regionally stimulate a single neural cell and record the potential signal of the nerve, and the detailed functions will be described hereinafter.

Accordingly, the present invention is directed to a nano-probe interface structure and a manufacturing method thereof, for applications in various neural prostheses.

The present invention is also directed to a nano-probe interface and a manufacturing method thereof, which is applicable for long-term effectively simulating or recording signals of the brain, so as to repair or partly replace the damaged tissues of sense organs.

To be embodied and broadly described herein, the present invention provides a nano-probe interface structure and a manufacturing method. The nano-probe interface structure at least includes a controller IC for neural cell recording and stimulation, a micro-electrode substrate array, and carbon nanotubes. The carbon nanotube can be a single-wall carbon nanotube, a double-wall carbon nanotube, a multi-wall carbon nanotube, a carbon nanotube bundle, a single, multiple, or carbon nanotube matrix. The controller IC is externally connected to the micro-electrode substrate array, and the carbon nanotube is located and grown on the micro-electrode array. If necessary, a thin isolation layer is coated around the carbon nanotube except only tips are exposed for conduction.

In the nano-probe interface structure and the manufacturing method thereof according to the embodiment of the present invention, the controller IC for neural cell recording and stimulation is an interface between a computer and a multifunctional nano-probe.

In the nano-probe interface structure and the manufacturing method thereof according to the embodiment of the present invention, the on-chip microfluidic channels are manufactured by a deep reactive ion etching (DRIE), the depths of the fluidic channels are about 200-500 μm, and the width of the fluidic channels is 10 μm at the minimum.

In the nano-probe interface structure and the manufacturing method thereof according to the embodiment of the present invention, the material of the conductive interconnects is conducting wires formed by boron or phosphorus dopant diffusion, the thickness of the conducting wires is controlled by a diffusion depth, and the impedance is adjusted accordingly based on the thickness of the conducting wires or dopant concentration.

In the nano-probe interface structure and the manufacturing method thereof according to the embodiment of the present invention, the diameter of the carbon nanotube is, for example, from 1 nm to 100 nm, and preferably from 1 nm to 50 nm.

In the nano-probe interface structure and the manufacturing method thereof according to the embodiment of the present invention, the thin isolation layer is, for examples, SiO₂, Al₂O₃, HfO₂, and ZrO₂.

In the nano-probe interface structure and the manufacturing method thereof according to the embodiment of the present invention, the thin isolation layer is, for example, SiO₂, and the thickness of the thin isolation layer is, for example, from 2 nm to 30 nm.

The present invention provides a method for manufacturing a nano-probe interface structure. In the method, a micro-electrode array is firstly provided, which has microfluidic channels and conductive interconnects. Next, a carbon nanotube is located and grown on the micro-electrode array. If necessary, a thin isolation layer is coated around the carbon nanotube except only tips are exposed for conduction. Afterwards, a controller IC for neural cell recording and stimulation is externally connected to the micro-electrode array.

In the method for manufacturing a nano-probe interface structure according to the embodiment of the present invention, the microfluidic channels are formed by defining a silicon dioxide layer and a silicon nitride layer on the silicon chip (100) through depositing and etching, so as to determine the microfluidic channels and the conductive interconnects. In a method for manufacturing the microfluidic channels, the microfluidic channels penetrating through the chip are manufactured by DRIE, and the depth of the fluidic channels is about 200-500 μm, and the width of the fluidic channels is 10 μm at the minimum. The conductive interconnects are formed by boron or phosphorus dopant diffusion, and the thickness of the conducting wires is controlled by the diffusion depth, and the impedance is adjusted accordingly based on the thickness of the conducting wires or dopant concentration.

In the method for manufacturing a nano-probe interface structure according to the embodiment of the present invention, the carbon nanotube is formed by chemical vapor deposition.

In the method for manufacturing a nano-probe interface structure according to the embodiment of the present invention, the temperature for forming the carbon nanotube is from 400° C. to 950° C., preferably from 700° C. to 950° C., the pressure is from 1 torr to 760 torr, and the introduced gas is a carbon-containing gas source, for examples, CH₄, C₂H₂, or C₂H₄, as well as H₂ and Ar.

In the method for manufacturing a nano-probe interface structure according to the embodiment of the present invention, the flow of CH₄ is from 1 sccm to 200 sccm.

In the method for manufacturing a nano-probe interface structure according to the embodiment of the present invention, the flow of H₂ is from 10 sccm to 100 sccm.

In the method for manufacturing a nano-probe interface structure according to the embodiment of the present invention, the flow of Ar is from 0 sccm to 400 sccm.

During the fabrication of the nano-probe interface structure of the present invention, the thin isolation layer for coating the outer wall of the carbon nanotube is prepared by sol-gel or chemical vapor atomic layer deposition (ALD), and the thickness of the thin isolation layer is, for example, from 2 nm to 30 nm.

In the present invention, a carbon nanotube can also be used as the probe of the nano-probe interface structure for neural recording and stimulation. As the carbon nanotube is at the nano-level in size and has a sufficient high mechanical strength, it can penetrate through the neural cell membrane without causing damages, so that the neural cell can survive for a relatively long time. Furthermore, the carbon nanotube has a high electrical conductivity to measure a trace amount of the potential difference transported by neural cells, so as to accurately and effectively enhance the reliability and long-term effectiveness of electrophysiological measurement.

In the present invention, a carbon nanotube with the outer wall being coated with a thin isolation layer can effectively avoid generating additional potential signals due to the mixing and electrical conduction of the electrophysiological solution with the solution in the neural cell after the puncturing process, thus the accuracy of electrophysiological measurement is enhanced.

In order to make the aforementioned and other aspects, features and advantages of the present invention comprehensible, preferred embodiments accompanied with figures are described in details below.

It is to be understood that both the foregoing general description and the following detailed description are exemplary, and are intended to provide further explanation of the invention as claimed.

### BRIEF DESCRIPTION OF THE DRAWINGS

The accompanying drawings are included to provide a further understanding of the invention, and are incorporated in and constitute a part of this specification. The drawings illustrate embodiments of the invention and, together with the description, serve to explain the principles of the invention.

FIGS. 1A to 1D are schematic cross-sectional views of several nano-probe interface structures according to an embodiment of the present invention.

FIG. 1E shows the result (example) of the nano-probe interface structure in the present invention obtained via the scanning electron microscope (SEM).

FIG. 2A is a schematic cross-sectional view of another nano-probe interface structure according to the embodiment of the present invention.

FIG. 2B is a schematic view of the nano-probe in FIG. 2A in performing neural cell recording and stimulation.

FIG. 3 is a schematic flow chart for manufacturing the nano-probe interface structure according to another embodiment of the present invention.

### DESCRIPTION OF EMBODIMENTS

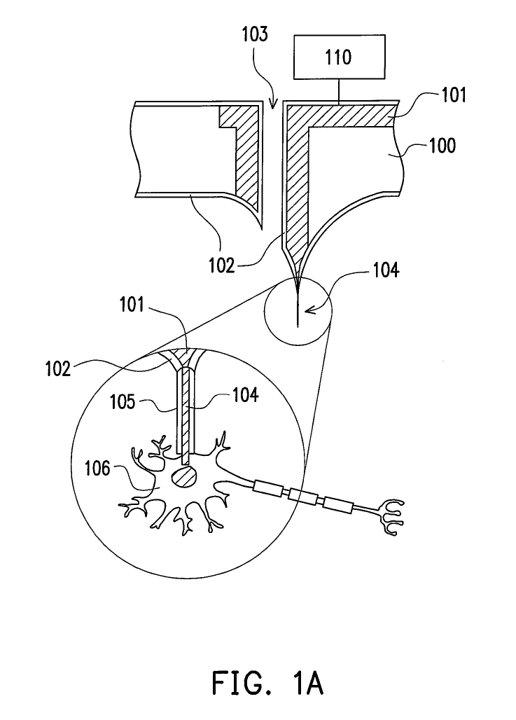

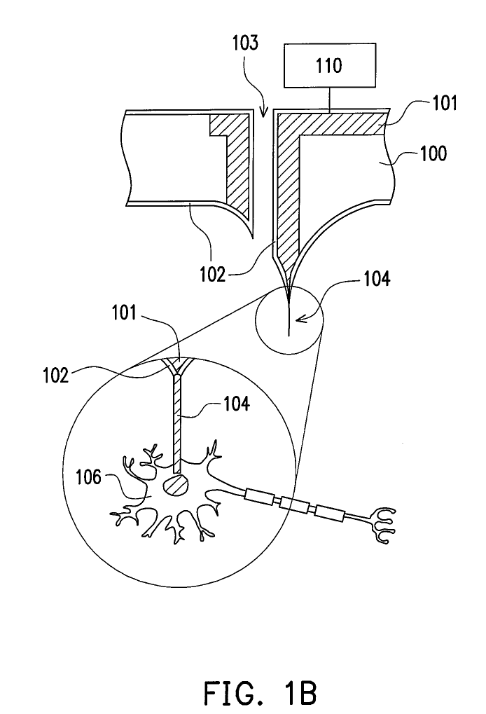

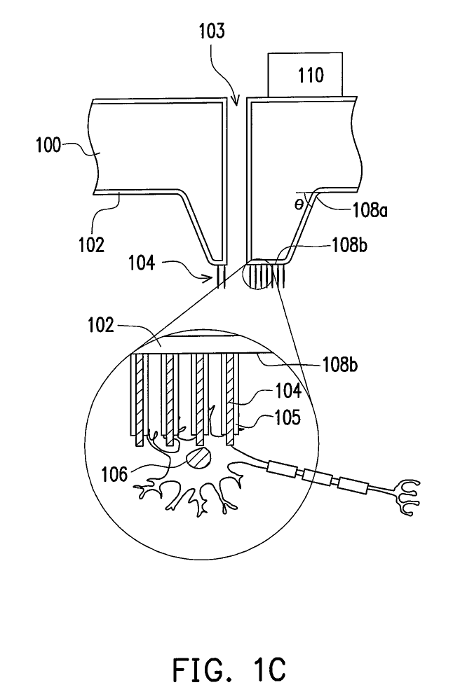

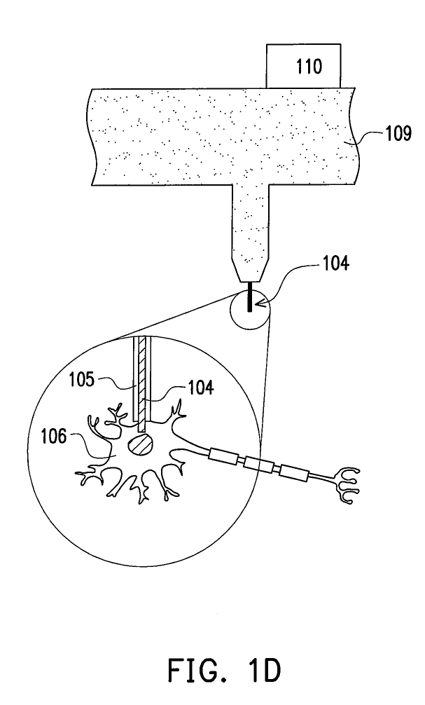

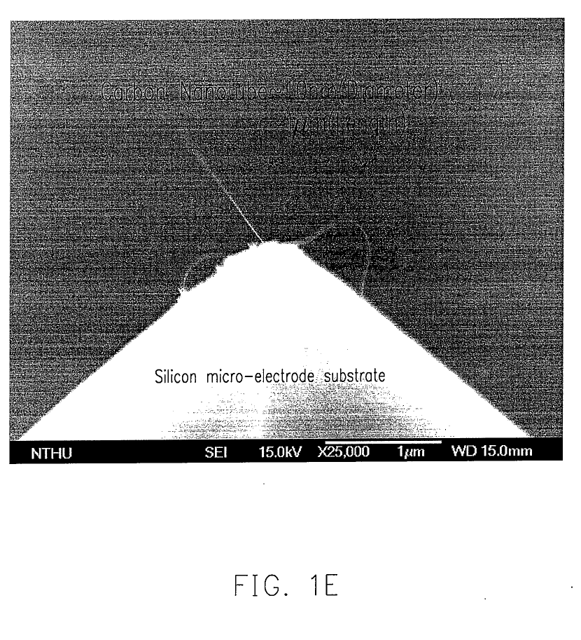

FIG. 1A is a schematic cross-sectional views of several nano-probe interface structures according to an embodiment of the present invention.

Referring to FIG. 1A, the nano-probe interface structure according to this embodiment at least includes a micro-electrode substrate array 100, a carbon nanotube 104, and a controller IC 110 for neural cell recording and stimulation. The carbon nanotube 104 is disposed on the micro-electrode substrate array 100. Though only one carbon nanotube is shown in FIG. 1A, in fact, the carbon nanotube 104 herein can be a single-wall carbon nanotube, a double-wall carbon nanotube, a multi-wall carbon nanotube, a carbon nanotube bundle, a single, multiple, or carbon nanotube matrix. The diameter of the carbon nanotube 104 is, for example, from 1 nm to 100 nm, and preferably from 1 nm to 50 nm. The controller IC 110 for neural cell recording and stimulation is externally connected to the micro-electrode array 100, and the controller IC 110 is the interface between a computer and a multifunctional nano-probe, which mainly aims at designing a recording and stimulation circuit for the microminiaturized nano-probe by the integrated circuit, so as to facilitate the implanting application in the future.

Referring to FIG. 1A, the micro-electrode array 100 has a microfluidic channel 103 penetrating through the micro-electrode array 100, and the depth of the microfluidic channel 103 is about 200-500 μm, and the width of the microfluidic channel 103 is merely 10 μm at the minimum. The microfluidic channel 103 can be used in the response of the neural cell upon various biochemical medicines and to realize the functions of supplying nutrition, delivering marker staining solution, or inducing growth, and so on. Additionally, the micro-electrode array 100 also has a conductive interconnect 101 therein, which can transport the potential signals measured by the carbon nanotube to an internal element. The material of the conductive interconnect 101 is a conducting wire formed by boron or phosphorus dopant diffusion, and the thickness of the conducting wires is controlled by the diffusion depth, and the impedance is adjusted accordingly based on the thickness of the conducting wires or dopant concentration. The conductive interconnect 101 has a borosilicate glass (BSG) isolation layer 102 being covered thereon for blocking noises.

Referring to FIG. 1A again, the carbon nanotube 104 further has a thin isolation layer 105 coated thereon, which is prepared by sol-gel or chemical vapor ALD. The thin isolation layer 105 is a dielectric layer, such as SiO₂, Al₂O₃, HfO₂, ZrO₂, and derivatives thereof, and the thickness of the thin isolation layer is, for example, from 2 nm to 30 nm.

In this embodiment, the nano-probe structure can be changed to perform puncturing process for various requirements or various functions. It is stated in the reference (see Sami Rosenblatt, Yuval Yaish, Jiwoong Park, Jeff Gore, Vera Sazonova, and Paul L. McEuen, Nano Lett., Vol. 2:869-872 (2002)) that, capacitance is generated on the surface of the carbon nanotube 104 due to the quantum effect, so the potential transfer between the interior and exterior of the neural cell membrane can be blocked, and the function of the thin isolation layer can also be achieved without coating an isolation layer. Referring to FIG. 1B, the carbon nanotube 104 is not coated with a thin isolation layer on the surface.

In this embodiment, the structure and shape of the silicon micro-electrode substrate array can be changed. As shown in FIG. 1C, the silicon micro-electrode substrate array has or does not have microfluidic channels 103; the angle 108a of the pointed cone of the array shape can be varied from 20° to 90° by changing the etching parameters, so as to have a pyramidal shape or columnar shape; the tip 108b of the pointed cone can be varied depending upon actual requirements, so as to change the area of the tip. The grown carbon nanotube is a single, multiple, or carbon nanotube array, for performing puncturing process for satisfying various requirements or achieving various functions.

Moreover, as the silicon micro-probe array has an insufficient elasticity, it is possible that the silicon micro-probe array is moved or falls off when the human body moves. Referring to FIG. 1D, the present invention also adopts a flexible substrate material 109 to replace the silicon micro-electrode substrate array for solving the problem. The flexible substrate material 109 is, for example, polylactic acid (PLA), polylactide-co-glycolide (PLGA).

FIG. 1E shows the result (example) of the nano-probe interface structure in the present invention obtained via the scanning electron microscope (SEM).

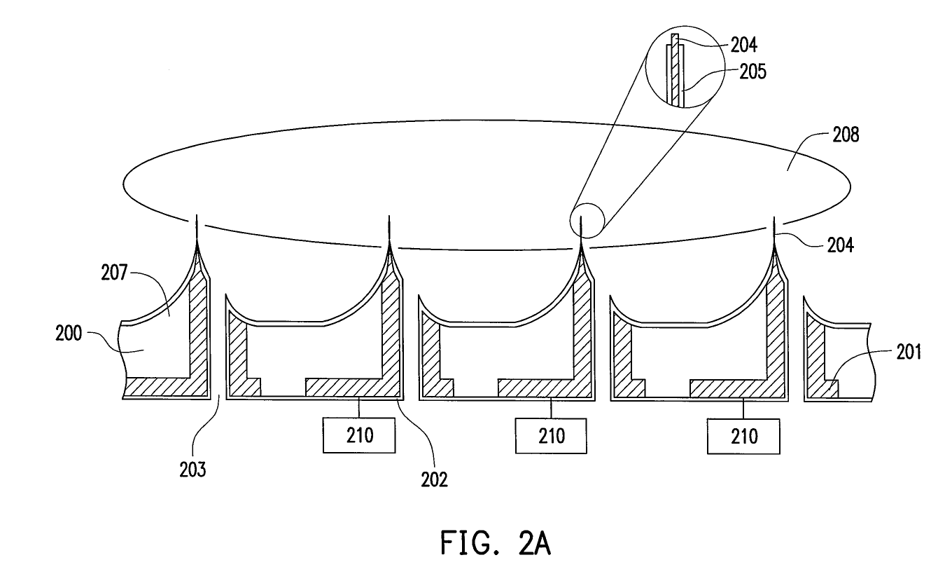

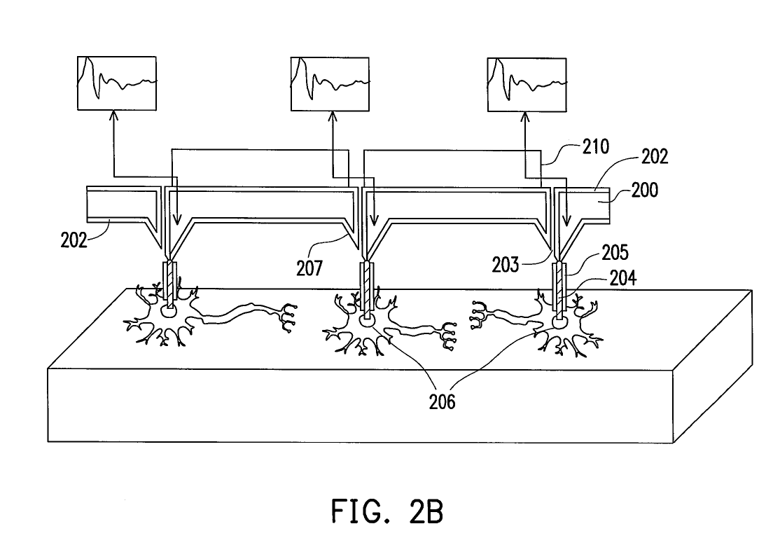

Referring to FIGS. 2A, 2B, the nano-probe array can stimulate a plurality of neural cells, reliably detect the activities of a plurality of neural cells 206, or be applied in stimulating and detecting the nerve biopsy 208, in which 200 indicates a micro-electrode substrate array, 201 indicates a conductive interconnect, 202 indicates borosilicate glass (BSG) isolation layer, 203 indicates a microfluidic channel, 204 indicates a carbon nanotube (single-wall, double-wall, multi-wall, bundles, single, multiple, or carbon nanotube matrix), 205 indicates an isolation layer, 207 indicates heavily doped silicon pointed cone substrate, and 210 indicates a controller IC for neural cell recording and stimulation.

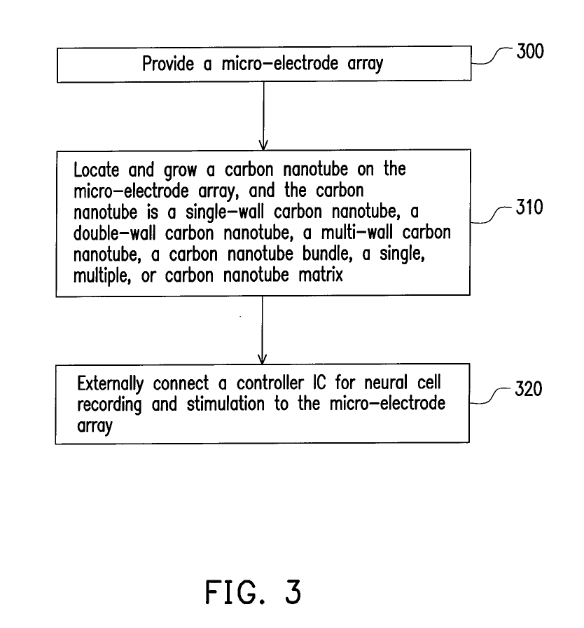

FIG. 3 is a schematic flow chart for manufacturing a nano-probe interface structure according to another embodiment of the present invention.

Referring to FIG. 3, firstly, a micro-electrode array is provided in Step 300, for example, microfluidic channels penetrating through a silicon substrate is manufactured in the silicon substrate by DRIE. The depth of the microfluidic channels is about 200-500 μm, and the width is merely 10 μm at the minimum. The microfluidic channels can be applied in the response of the neural cell upon various biochemical medicines and to realize the functions of supplying nutrition, delivering marker staining solution, or inducing growth, and so on. Additionally, the micro-electrode array also has conductive interconnects for transporting the potential signals measured by the carbon nanotube to an internal element. The conductive interconnects can be formed by boron or phosphorus dopant diffusion, and the thickness of the conducting wires is controlled by the diffusion depth, and the impedance is adjusted accordingly based on the thickness of the conducting wires or dopant concentration. Additionally, the conductive interconnects can have a borosilicate glass (BSG) isolation layer coated thereon for blocking noises.

Next, in Step 310, a carbon nanotube is located and grown on the micro-electrode substrate array, and the carbon nanotube is a single-wall carbon nanotube, a double-wall carbon nanotube, a multi-wall carbon nanotube, a carbon nanotube bundle, a single, multiple, or carbon nanotube matrix. During the process for manufacturing the nano-probe interface structure of the present invention, a thick photoresist is uniformly spin-coated on the micro-electrode array by a spin coater, and the photoresist on the conductive interconnects is removed by a mini probe or by etching. Then, a catalyst, such as iron, cobalt, or nickel, is evaporated on the micro-probe array by an electron gun. Afterwards, the photoresist is removed by the lift-off process; or iron, cobalt, or nickel, for example, is imprinted on the micro-probe array by the nano-imprint technique, and merely the catalyst on the conductive interconnects in the micro-electrode substrate array is remained. Finally, a carbon nanotube is located and grown. This process is simple and can successfully locate and grow the carbon nanotube. The carbon nanotube is formed at a temperature, for example, from 400° C. to 950° C., preferably from 700° C. to 950° C., and at a pressure, for example, from 1 torr to 760 torr, and the introduced gases are, for examples, CH₄, H₂, and Ar. The flow of CH₄, which also can be a carbon-containing source, such as C₂H₂ and C₂H₄, for example, is from 1 sccm to 200 sccm. The flow of H₂ is, for example, from 10 sccm to 100 sccm, and the flow of Ar is, for example, from 0 sccm to 400 sccm. The diameter of the carbon nanotube 104 is, for example, from 1 nm to 100 n, and preferably from 1 nm to 50 nm.

Afterwards, between Step 310 and the subsequent Step 320, a thin isolation layer, which is prepared, for example, by sol-gel or chemical vapor ALD, can be coated on the outer wall of the carbon nanotube after location and growth.

Finally, in Step 320, a controller IC for neural cell recording and stimulation is externally connected to the micro-electrode substrate array. The controller IC is an interface between a computer and a multifunctional nano-probe, which aims at providing a recording and a stimulation circuits for the nano-probe to facilitate the implanting applications in the future.

In view of the above, in the nano-probe interface structure of the present invention, a carbon nanotube is directly located and grown on the micro-electrode substrate array. The carbon nanotube has a size of less than 1 nm-50 nm, which is much smaller than neural cells in the micron scale. When puncturing neural cells, the carbon nanotube can effectively prolong the survival time of a neural cell, and enhance the variability of the electrophysiological experiments. Additionally, by taking the carbon nanotube having a high conductivity as a nano-probe wire, and taking the material having a thin isolation layer coated on the outer wall as the isolation layer, the electrical leakage can be effectively reduced and the sensitivity of the nano-probe interface element can be effectively enhanced.

It will be apparent to those skilled in the art that various modifications and variations can be made to the structure of the present invention without departing from the scope or spirit of the invention. In view of the foregoing, it is intended that the present invention cover modifications and variations of this invention provided they fall within the scope of the following claims and their equivalents.

What is claimed is:

1. A nano-probe interface structure, comprising:
a micro-electrode array;
carbon nanotubes, disposed on the micro-electrode array, wherein the carbon nanotubes are single-wall carbon nanotubes, double-wall carbon nanotubes, multi-wall carbon nanotubes, a carbon nanotube bundle, a single, multiple, or carbon nanotube matrix; and
a controller IC, connected to the micro-electrode substrate array for neural cell recording and stimulation.

2. The nano-probe interface structure as claimed in claim 1, wherein the controller IC for neural cell recording and stimulation serves as an interface between a computer and the multifunctional nano-probe.

3. The nano-probe interface structure as claimed in claim 1, wherein the micro-electrode array is a silicon micro-electrode substrate array.

4. The nano-probe interface structure as claimed in claim 1, wherein the micro-electrode array comprises pointed cones with microfluidic channels or pointed cones without microfluidic channels, the pointed cone is a pyramidal or columnar shape according to the variation of etching angle, and the area of the tip of the pointed cone varies depending upon actual requirements.

5. The nano-probe interface structure as claimed in claim 1, wherein the micro-electrode array is made of silicon micro-electrodes or a flexible substrate material.

6. The nano-probe interface structure as claimed in claim 5, wherein the flexible substrate material comprises polylactic acid (PLA) or polylactide-co-glycolide (PLGA).

7. The nano-probe interface structure as claimed in claim 1, wherein the diameter of the carbon nanotube is from 1 nm to 100 nm.

8. The nano-probe interface structure as claimed in claim 1, further comprising a thin isolation layer coated around the carbon nanotube, for enhancing the sensitivity, wherein the thickness of the thin isolation layer is from 2 nm to 30 nm n.

9. The nano-probe interface structure as claimed in claim 8, wherein the thin isolation layer is made of SiO₂, Al₂O₃, HfO₂, or ZrO₂.

10. The nano-probe interface structure as claimed in claim 1, wherein the micro-electrode array further comprises microfluidic channels and conductive interconnects.

11. The nano-probe interface structure as claimed in claim 10, wherein the depth of the microfluidic channels is about 200-500 μm, and the width is 10 μm at the minimum.

12. The nano-probe interface structure as claimed in claim 10, wherein the material of the conductive interconnects is a conducting wire formed by boron or phosphorus dopant diffusion, and the thickness of the conducting wires is controlled by the diffusion depth, and the impedance is adjusted accordingly based on the thickness of the conducting wires or dopant concentration.

13. A method for manufacturing a nano-probe interface structure, comprising:
providing a micro-electrode substrate array;
locating and growing a carbon nanotube on the micro-electrode substrate array, wherein the carbon nanotube is a single-wall carbon nanotube, a double-wall carbon nanotube, a multi-wall carbon nanotube, a carbon nanotube bundle, a single, multiple, or carbon nanotube matrix; and
externally connecting a controller IC for neural cell recording and stimulation to the micro-electrode substrate array.

14. The method for manufacturing the nano-probe interface structure as claimed in claim 13, wherein the process for providing the micro-electrode array comprises providing a micro-electrode array having microfluidic channels and conductive interconnects.

15. The method for manufacturing the nano-probe interface structure as claimed in claim 14, wherein the microfluidic channels are manufactured by means of deep reactive ion etching (DRIE).

16. The method for manufacturing the nano-probe interface structure as claimed in claim 14, wherein the conductive interconnects are formed by or phosphorus dopant diffusion, and the thickness of the conducting wires is controlled by the diffusion depth, and the impedance is adjusted accordingly based on the thickness of the conducting wires or dopant concentration.

17. The method for manufacturing the nano-probe interface structure as claimed in claim 13, wherein the process for locating and growing the carbon nanotube comprises a self-assembly method.

18. The method for manufacturing the nano-probe interface structure as claimed in claim 13, wherein the process for locating and growing the carbon nanotube comprises a chemical vapor deposition.

19. The method for manufacturing the nano-probe interface structure as claimed in claim 18, wherein the temperature for locating and growing the carbon nanotube is from 400° C. to 950° C., and the pressure is from 1 torr to 760 torr, and an introduced gas comprises CH₄, C₂H₂, or C₂H₄, as well as H₂ and Ar.

20. The method for manufacturing the nano-probe interface structure as claimed in claim 19, wherein the flow of CH₄ is from 1 sccm to 200 sccm.

21. The method for manufacturing the nano-probe interface structure as claimed in claim 19, wherein the flow of H₂ is from 10 sccm to 100 sccm.

22. The method for manufacturing the nano-probe interface structure as claimed in claim 19, wherein the flow of Ar is from 0 sccm to 400 sccm.

23. The method for manufacturing the nano-probe interface structure as claimed in claim 13, further comprising coating a thin isolation layer around the carbon nanotube.

24. The method for manufacturing the nano-probe interface structure as claimed in claim 23, wherein the thin isolation layer is prepared by a sol-gel chemical deposition or a chemical vapor atomic layer deposition (ALD).
